(19) United States
(12) Patent Application Publication
(10) Pub. No.: US 2024/0100208 A1
(43) Pub. Date: Mar. 28, 2024

(54) INNOVATIVE DISINFECTION AND STERILIZATION EQUIPMENT SYSTEM

(71) Applicant: Jiangsu Ruizhikang Intelligent Technology Development Co., Ltd., Wuxi City (CN)

(72) Inventors: Pengzhou LEI, Wuxi City (CN); Yihao ZHANG, Wuxi City (CN); Lingli SUN, Wuxi City (CN); Junhong CHEN, Wuxi City (CN); Yingqiang ZHAO, Wuxi City (CN); Bo WANG, Wuxi City (CN); Xiangye ZENG, Wuxi City (CN); Zhanjun ZHANG, Wuxi City (CN); Jiandong ZHANG, Wuxi City (CN); Zhiqiang SHI, Wuxi City (CN)

(21) Appl. No.: 17/982,256

(22) Filed: Nov. 7, 2022

(30) Foreign Application Priority Data
Sep. 28, 2022 (CN) 2022111961350

(51) Int. Cl. A61L 2/18; A61L 2/24; A61L 2/26; C02F 1/00; C02F 1/467

(52) U.S. Cl. CPC A61L 2/183; A61L 2/24; A61L 2/26; C02F 1/001; C02F 1/4672; C02F 2303/04

(57) ABSTRACT

An innovative equipment system comprises a tap water reservoir system, a disinfectant reactor system, a pump system, a disinfection and safety door spraying system, a sewage recycling system and a sewage treatment system, wherein the tap water reservoir system is mainly configured to store tap water in advance according to actual cargo volumes and disinfection requirements, and generally in tons; and the disinfectant reactor system comprises boron-doped diamond electrode modules, a reactor indicating lamp module, a visible reactor, a smart control screen, at least one water tank and a structure protection module. The Innovative disinfection and sterilization equipment system can easily kill bacteria and viruses such as Escherichia coli, Staphylococcus aureus, Salmonella typhimurium, and new coronavirus in a short time and enhance disinfection efficiency. The present invention has no corrosive or irritating effect and is environmental-friendly that would not cause harm to human bodies and would not corrode metal surfaces.

### TECHNICAL FIELD

The present invention relates to the technical field of disinfection and sterilization equipment, in particular to an innovative disinfection and sterilization equipment system.

### Background Technology

In response to the severe and complex foreign new coronavirus pandemic and the frequent occurrence of viruses of other types abroad, Beijing CDC released the “Technical Guidelines for the Prevention and Control of International Mails and Express Mails During the Epidemic of New Coronary Pneumonia (First Edition)”, under which, when international mails enter the Chinese customs, the new coronavirus detection and monitoring shall be carried out according to the regulations, and comprehensive preventive disinfection shall be conducted on imported mail bags and outer packaging of express items after opening the bags; with regard to international express mails, customs supervision centers, ports, sorting centers and terminal outlets shall use 500 mg/L chlorine-containing disinfectants or other disinfectants that can be employed on surfaces to spray and sanitize all surfaces of outer packaging of each mail piece, and the surfaces shall get covered and wet completely with disinfectants being retained thereon for at least 30 minutes. The same applies to similar scenarios such as highway entrances and exits, entrances and exits of industrial parks, entrances and exits of farms and garbage stations in COVID-19 high-incidence areas.

At present, disinfectants are often utilized in chemical industries and are flammable, which require special handling and control during storage and transport; further disinfectants are highly corrosive and irritating to human eyes, skin, metals and so on; especially chemical disinfectants are consumables that need to be constantly repurchased in time; in addition, even most commonly used disinfectants leave significant residues that will harm and pollute the environment to some extent, and cannot be discharged via ordinary sewage channels. Therefore, it is urgent to propose an innovative disinfection and sterilization equipment system to solve technical problems mentioned above.

### SUMMARY OF THE INVENTION

In order to address the technical problems mentioned in the background technology, the present invention provides an innovative disinfection and sterilization equipment system.

The present invention has following technical solutions to solve above-mentioned technical problems. An innovative disinfection and sterilization equipment system, comprises a tap water reservoir system, a disinfectant reactor system, a disinfection and safety door spraying system, a sewage recycling system and a sewage treatment system;
the tap water reservoir system is mainly configured to store tap water in advance according to actual cargo volumes and disinfection requirements, and generally in tons;
the disinfectant reactor system comprises boron-doped diamond (BDD) electrode modules, a reactor indicating lamp module, a visible reactor, a smart control screen, at least one water tank and a structure protection module;
the water pump system collects bactericidal ozone water flowing out of the disinfectant reactor system quickly into the disinfection and safety door spraying system, and increases pressure of the bactericidal ozone water after pressurizing by water pumps;
the disinfection and safety door spraying system comprises indicating doorplates, inlet pipes, branch water pipes, a spraying system, a speed bump, a return water groove and a lifting and limiting module;
the sewage recycling system is configured to collect and recycle sewage through the return water groove provided at a bottom portion of the disinfection and safety door spraying system via pipes, and also arranged to collect rainwater; and
the sewage treatment system has two implementation schemes according to actual needs, a general sewage purification scheme and a high-level sewage purification scheme.

The innovative disinfection and sterilization equipment system of the present invention can carry out 360-degree sterilization spray for vehicles passing through the customs, highway entrances and exits, industrial park entrances and exits, farm entrances and exits, or garbage collection station entrances and exits within 3 minutes and with a sterilization rate over 99%; and the present invention achieves better disinfection and sterilization effectiveness because ozone in water has a half-life of 0.5 h and when passing through the disinfection and safety door spraying system, vehicles can be continually disinfected in at least 20 minutes.

Preferably, water treated by the sewage recycling system is required to have a pH value below 7.5.

Preferably, the general sewage purification scheme is designed only for recycled sewage, wherein garbage of large size such as leaves and stones fallen from vehicles is filtered by a filter system, after judgment by a water quality particle sensor, water is returned to the at least one water tank after precipitation for specific time and dusts and foreign matters are precipitated.

Preferably, the high-level sewage purification scheme is designed for recycled filtered sewage, specifically, garbage of large particles such as leaves, stones, etc., washed or fallen from freight vehicles are filtered through a filter system, and after judgment by a water quality particle sensor, precipitate for a specific time duration, after precipitation, ionize water molecules by at least two 4-inch BDD diamond membrane electrode modules or a plurality of small-size BDD diamond membrane electrode modules energized at low voltage (within 36V) and current of each pair of membranes more than 20 A, thus ozone water is generated to treat sewage to achieve sewage purification with a high cleanliness level.

A speed bump (or a speed limit sign) could be arranged in front of the present invention, so that drivers could slow vehicles down before entering into a disinfection area; size and position sensors could be provided to measure basic information of vehicles and goods like sizes of container trucks, based on which volumes of spray could be determined before starting the disinfection and sterilization equipment system of the present invention; inside the disinfection and safety door spraying system are provided disinfection spray nozzles, respectively arranged at four directions, up, down, left and right sides thereof, so as to spray evenly and increase disinfection efficiency; and the ozone water is sprayed in 360 degrees to ensure complete disinfection, and when disinfection is finished and vehicles depart from exits, infrared cameras could be arranged at exits to record disinfected vehicles, time information and so on.

Compared to the prior art, the present invention has following beneficial effects:

Firstly, the present invention can easily kill bacteria and viruses such as Escherichia coli, Staphylococcus aureus, Salmonella typhimurium, and new coronavirus in a short time, and achieves improved disinfection efficiency.

Secondly, instead of adopting chemical disinfectants, the present invention accomplishes disinfection and sterilization merely by means of adding some water which is easier to obtain.

Thirdly, the present invention makes no corrosive or irritating effect and is so environmentally friendly that would not cause harm to human bodies and would not corrode metal surfaces

Fourthly, the system of present invention can be used repeatedly, which is a one-time investment configuration and hardly needs follow-up costs.

Lastly, the present invention achieves remarkable disinfection and sterilization performance with no emission nor pollution, and is provided with a sewage recycling system so that water can be recycled and reused.

### BRIEF DESCRIPTION OF THE DRAWINGS

FIG. 1 is a schematic diagram of an innovative disinfection and sterilization equipment system of the present invention;

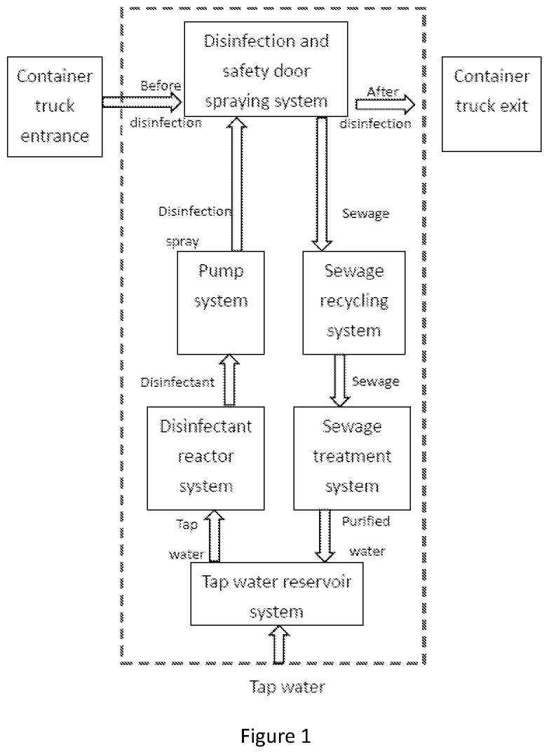

FIG. 2 is a flow chart of a spraying system of the present invention; and

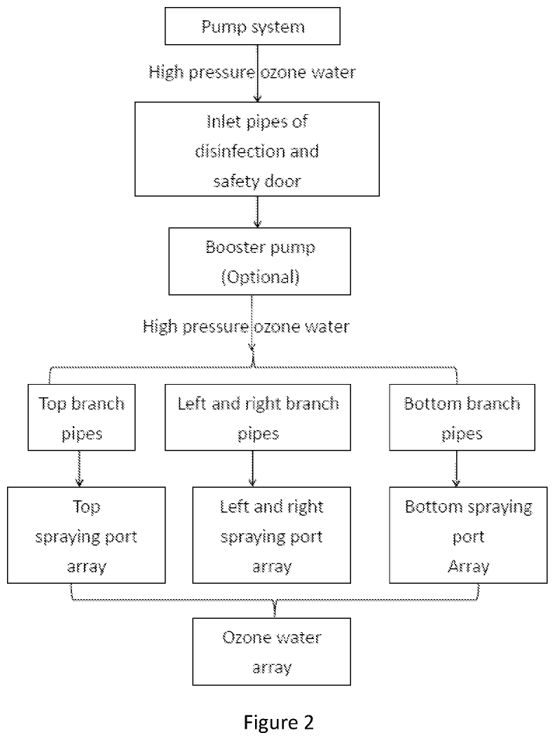

FIG. 3 is a flow chart of a sewage recycling system of the present invention.

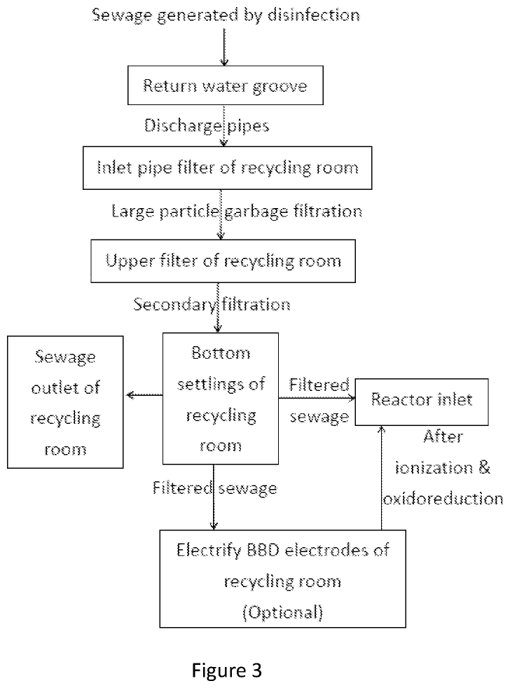

### SPECIFIC EMBODIMENTS

The technical solutions of embodiments of the present invention will be clearly and completely described below in conjunction with attached drawings in the embodiment of the present invention. Clearly, the described embodiments are only some of embodiments of the present invention rather than all of them. Based on the embodiments described in the present invention, all other embodiments obtained by ordinary technicians in the field without creative labor shall fall within protection scope of the present invention.

### Embodiment 1

The present embodiment is a first specific implementation of the innovative disinfection and sterilization equipment system of the present invention.

An innovative disinfection and sterilization equipment system, comprises a tap water reservoir system, a disinfectant reactor system, a disinfection and safety door spraying system, a sewage recycling system and a sewage treatment system;
the tap water reservoir system is mainly configured to store tap water in advance according to actual cargo volumes and disinfection requirements, and generally in tons;
the disinfectant reactor system comprises boron-doped diamond electrode modules, a reactor indicating lamp module, a visible reactor, a smart control screen, at least one water tank and a structure protection module;
the water pump system collects bactericidal ozone water flowing out of the disinfectant reactor system quickly into the disinfection and safety door spraying system, and increases pressure of the bactericidal ozone water after pressurizing by water pumps;
the disinfection and safety door spraying system comprises indicating doorplates, inlet pipes, branch water pipes, a spraying system, a speed bump, a return water groove and a lifting and limiting module;
the sewage recycling system is configured to collect and recycle sewage through the return water groove provided at a bottom portion of the disinfection and safety door spraying system via pipes, and also arranged to collect rainwater; and the sewage treatment system has two implementation schemes according to actual needs, a general sewage purification scheme and a high-level sewage purification scheme.

Specifically, water treated by the sewage treatment system is required to have a pH value below 7.5.

Reactions on surfaces of the BDD (boron-doped diamond) electrodes are depicted as following formulas:

BDD+H2O BDD(OH)+H++e−;

BDD(OH)+R BDD+mCO2+nH2O; and

BDD(OH) BDD+mCO2+H++e−.

Specifically, the general sewage purification scheme is designed only for recycled sewage, wherein garbage of large size such as leaves and stones fallen from vehicles is filtered by a filter system, after judgment by a water quality particle sensor, water is returned to the at least one water tank after precipitation for specific time and dusts and foreign matters are precipitated.

Specifically, the high-level sewage purification scheme is designed for recycled filtered sewage, specifically, garbage of large particles such as leaves, stones, etc., washed or fallen from freight vehicles are filtered through a filter system, and after judgment by a water quality particle sensor, precipitate for a specific time duration, after precipitation, ionize water molecules by at least two 4-inch BDD diamond membrane electrode modules or a plurality of small-size BDD diamond membrane electrode modules energized at low voltage (within 36V) and current of each pair of membranes more than 20 A, thus ozone water is generated to treat sewage to achieve sewage purification with a high cleanliness level.

### Embodiment 2

The present embodiment is a second specific implementation of the innovative disinfection and sterilization equipment system of the present invention.

A disinfectant reactor system of the innovative disinfection and sterilization equipment system comprises boron-doped diamond electrode modules, a reactor indicating lamp module, a visible reactor, a smart control screen, at least one water tank and a structure protection module.

By virtue of the above technical solutions, tap water offered by the reservoir system is collected into water tank, the BDD (boron-doped diamond) electrode modules arranged bottom or inner portions of the water tank are electrified at a low voltage (within 36V) to achieve ionization of water molecules and generate ozone water; a reactor indicating lamp module shows different operational states of the machine through indicating lamps of different colors; inside the visible reactor is arranged a sampling concentration detector so that when a concentration is lower than a preset value, the disinfectant reactor system will be automatically started, and operations will be automatically stopped when an expected concentration is reached, the smart control screen is arranged outside the visible reactor and can be customized to display and set different information, such as real-time display of operations of internal devices, display and setting of a flow rate of water supply, and sampling water concentration required for disinfection, display of information of vehicles that have been disinfected, display of diamond module usage and life and so on; and the structure protection module can not only support and protect internal modules, appearance thereof can be customized.

### Embodiment 3

The present embodiment is a third specific implementation of the innovative disinfection and sterilization equipment system of the present invention.

The disinfection and safety door spraying system comprises indicating doorplates, inlet pipes, branch water pipes, a spraying system, a speed bump, a return water groove and a lifting and limiting module.

By dint of the above technical solutions, the indicating doorplates can be customized with different texts or patterns, the ozone water pressurized by the pump system is delivered to the inlet pipes of the disinfection and safety door spraying system through pipes, and flows into the branch pipes; the spraying system comprises spray nozzles respectively arranged at top, left, right and bottom portions thereof, the spray nozzles of refined sizes (with diameters below 2 mm) cooperates with water pumps under high pressure to generate ozone water mist, and within every 50 cm is arranged a spray nozzle, so as to ensure uniform spray and 360-degree disinfection without any blind angle, and the rated flow is 25 L/min and above; the speed bump further enables vehicles to be decelerated to 5 km/h or below, so as to ensure the sterilizing water mist is sprayed evenly, thereby guarantying good disinfection effect; and after disinfection and sterilization the return tank introduces the sewage into the sewage treatment system.

It should be noted that, in this document, relational terms such as first and second are only used to distinguish one entity or operation from another, and it is not necessary to advise or imply any such actual relationship or sequence exists between these entities or operations. Moreover, the terms like “comprising” and “including” or any other variants thereof are intended to cover non-exclusive inclusions, such that, a process, method, article or apparatus comprising a series of elements includes not only those elements, but also other elements not expressly listed, or elements inherent to such process, method, article or apparatus.

At last, it shall be clear that the foregoing are only preferred embodiments of the present Invention which are not intended to limit the present invention. Notwithstanding the detailed description of the present invention with reference to the foregoing embodiments are made, it is still possible for a technician in the field to modify the technical solutions recorded in each of the foregoing embodiments, or to equivalently replace some of the technical features thereof. Any modification, equivalent substitution, improvement, etc. made within the spirit and principles of the present invention shall be included within scope of protection of the present invention.

1. An innovative disinfection and sterilization equipment system, comprising a tap water reservoir system, a disinfectant reactor system, a disinfection and safety door spraying system, a sewage recycling system and a sewage treatment system, wherein
the tap water reservoir system is configured to store tap water in advance according to actual cargo volumes and disinfection requirements, and generally in tons;
the disinfectant reactor system comprises boron-doped diamond electrode modules, a reactor indicating lamp module, a smart control screen, at least one water tank and a structure protection module;
the water pump system collects bactericidal ozone water flowing out of the disinfectant reactor system quickly into the disinfection and safety door spraying system, and increases pressure of the bactericidal ozone water after pressurizing by water pumps;
the disinfection and safety door spraying system comprises indicating doorplates, inlet pipes, branch water pipes, a spraying system, a speed bump, a return water groove and a lifting and limiting module;
the sewage recycling system is configured to collect and recycle sewage through the return water groove provided at a bottom portion of the disinfection and safety door spraying system via pipes, and also arranged to collect rainwater; and
the sewage treatment system has two implementation schemes according to actual needs, a general sewage purification scheme and a high-level sewage purification scheme.

2. The innovative disinfection and sterilization equipment system according to claim 1, wherein water treated by the sewage recycling system is required to have a pH value below 7.5.

3. The innovative disinfection and sterilization equipment system according to claim 1, wherein the general sewage purification scheme is designed only for recycled sewage, wherein garbage of large size such as leaves and stones fallen from vehicles is filtered by a filter system, after judgment by a water quality particle sensor, water is returned to the at least one water tank after precipitation for specific time and dusts and foreign matters are precipitated.

4. The innovative disinfection and sterilization equipment system according to claim 1, wherein the high-level sewage purification scheme is designed for recycled filtered sewage, specifically, garbage of large particles such as leaves, stones, etc., washed or fallen from freight vehicles are filtered through a filter system, and after judgment by a water quality particle sensor, precipitate for a specific time duration, after precipitation, ionize water molecules by at least two 4-inch BDD diamond membrane electrode modules or a plurality of small-size BDD diamond membrane electrode modules energized at low voltage (within 36V) and current of each pair of membranes more than 20 A, thus ozone water is generated to treat sewage to achieve sewage purification with a high cleanliness level.
